(19) United States
(12) Patent Application Publication
(10) Pub. No.: US 2003/0091465 A1
(43) Pub. Date: May 15, 2003

(54) Multi-layer deodorizing device and method of deodorization

(76) Inventor: Amy Hendricks, Monroe, GA (US)

Correspondence Address: MYERS & KAPLAN, INTELLECTUAL, PROPERTY LAW, L.L.C., 1827 POWERS FERRY ROAD, BUILDING 3, SUITE 200,, ATLANTA, GA, 30339, US

(21) Appl. No.: 10/236,011

(22) Filed: Sep. 5, 2002

Related U.S. Application Data
(60) Provisional application No. 60/317,218, filed on Sep. 5, 2001.

(51) Int. Cl. A61L 9/12

(52) U.S. Cl. 422/5; 422/122; 422/123; 239/36; 236/53

(57) ABSTRACT

A multi-layer deodorizing device having at least one generally flat, fragrance-infused layer enabling cloaking of unpleasant odors, at least one odor-absorbent layer enabling substantial removal of malodors from a surface or personal article, and a specialized shape enabling deodorization of a targeted personal article, such as for exemplary purposes only, a shoe, wherein an individual desires to utilize a temporary storage period to remove odors and refresh a personal article, such as a shoe.

### CROSS-REFERENCE TO RELATED APPLICATION

To the full extent permitted by law, the present application claims priority to and the benefit as a non-provisional application to provisional patent application entitled “Multi-layer Deodorizing Device and Method of Deodorization” filed on Sep. 5, 2001, having assigned Serial No. 60/317,218, wherein said application is incorporated herein by reference.

### TECHNICAL FIELD

The present invention relates generally to odor control devices and, more specifically, to a multi-layer deodorizing device and method for deodorizing personal articles and spaces. The present invention is particularly useful in, although not strictly limited to, footwear applications wherein an individual desires to remove odors and enjoy a refreshed shoe by placing a multi-layer deodorizing device therein during storage and by removing the device to enable wearing of the deodorized shoe.

### BACKGROUND OF THE INVENTION

Most individuals have experienced unpleasant foot or shoe odors. Soil and perspiration contaminate shoes and, as the interior surfaces become saturated, odors can be emitted. Athletic shoes, whether for running, organized team sports, golf or weight training, are routinely subjected to sweaty conditions. As a result, these types of shoes can quickly become undesirably aromatic.

Other types of shoes can also absorb and generate odors. Sometimes the leather, fabric or artificial surfaces of the shoe itself contribute to or exacerbate odor issues. Even open shoes and sandals can influence the air in a closet or storage container, thereby affecting the freshness of the other articles stored therewith. As a result, the marketplace offers a variety of products directed toward the control unpleasant odors.

An ever-increasing variety of home air freshener devices continue to be introduced. Many of these require plugging into an electric outlet, and are thus often disadvantageously limited from utilization in a closet or storage container. Others are free-standing and able to be used in small places. Although these independently functioning air fresheners can positively influence the available air in a closet or storage container through the use of fragrance, each remains disadvantageously unable to directly affect or remove the odors within a shoe or other substantially enclosed article.

Some devices have been suggested to address shoe and foot odors specifically. Fragranced powders may be sprinkled on a foot or in a shoe in an attempt to prevent odors from forming. Unfortunately, not only are odors still able to be generated, but the powders can clump uncomfortably, or can ruin the interior surfaces of the shoe. Powder receptacles have also been suggested, wherein an individual fills the receptacle and places it into a shoe while the shoe is stored. These filled receptacles do assist with masking some odors, however they are unable to come into direct contact with the majority of the interior surface of a shoe and are thus unable to remove odors therefrom. Each of the foregoing approaches is disadvantageous in view of the present invention.

Therefore, it is readily apparent that there is a need for a deodorizing device having a fragrance feature enabling masking of unpleasant odors, an odor-absorbent feature enabling removal of unpleasant odors, and a selected shape enabling effective deodorization of a targeted personal article, such as for exemplary purposes only, a shoe, thus preventing the above-discussed disadvantages.

### BRIEF SUMMARY OF THE INVENTION

Briefly described, in a preferred embodiment, the present invention overcomes the above-mentioned disadvantages, and meets the recognized need for such a device, by providing a multi-layer deodorizing device and method for deodorizing personal articles and spaces, wherein malodors are masked and eliminated.

According to its major aspects and broadly stated, the present invention is a multi-layer deodorizing device having at least one generally flat, fragrance-infused layer enabling cloaking of unpleasant odors, at least one odor-absorbent layer enabling substantial removal of malodors from a surface or personal article, and a specialized shape enabling deodorization of a targeted personal article, such as for exemplary purposes only, a shoe, wherein an individual desires to utilize a temporary storage period to remove odors and refresh a personal article, such as a shoe.

More specifically, the present invention is a substantially flat multi-layer deodorizer having a first layer formed from generally fibrous material such as, for exemplary purposes only, filter paper, felt, blotter paper or cardboard and a second layer formed from generally porous material such as, for exemplary purposes only, charcoal. One skilled in the art would readily recognize that alternative layer configurations could be utilized such as, for exemplary purposes only, additional layers of fibrous material or porous material or combinations thereof.

The fibrous layer is impregnated and infused with fragrance. The porous layer, preferably charcoal, is permanently affixed to the fibrous layer, thereby forming a multi-layer device. The multi-layer deodorizer is cut or otherwise formed into a desired size and patterned such as, for exemplary purposes only, a shoe silhouette. The dimensions of the multi-layer deodorizer are defined to enable placement thereof into a specific type or size of personal article or accessory.

A feature and advantage of the present invention is the ability of such a device to provide assistance in the deodorizing of personal articles or accessories, such as shoes.

A feature and advantage of the present invention is the ability of such a device to provide a specialized tool for deodorizing personal articles and accessories.

A feature and advantage of the present invention is the ability of such a device to provide a disposable deodorizer, thereby extending the useful life of personal articles and accessories, such as shoes.

A feature and advantage of the present invention is the ability of such a device to enable efficient deodorizing and freshening of substantially closed storage spaces.

A feature and advantage of the present invention is the ability of such a device to effectively mate with the interior area of a shoe.

These and other objects, features and advantages of the invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a perspective view of a multi-layer deodorizing apparatus according to a preferred embodiment of the present invention, showing a device shape targeted to fit into a woman's shoe.

FIG. 2 is a perspective view of a multi-layer deodorizing apparatus according to a preferred embodiment of the present invention, showing a device shape targeted to fit into man's shoe.

### DETAILED DESCRIPTION OF THE PREFERRED AND ALTERNATE EMBODIMENTS

In describing the preferred and alternate embodiments of the present invention, as illustrated in the figures, specific terminology is employed for the sake of clarity. The invention, however, is not intended to be limited to the specific terminology so selected, and it is to be understood that each specific element includes all technical equivalents that operate in a similar manner to accomplish similar functions.

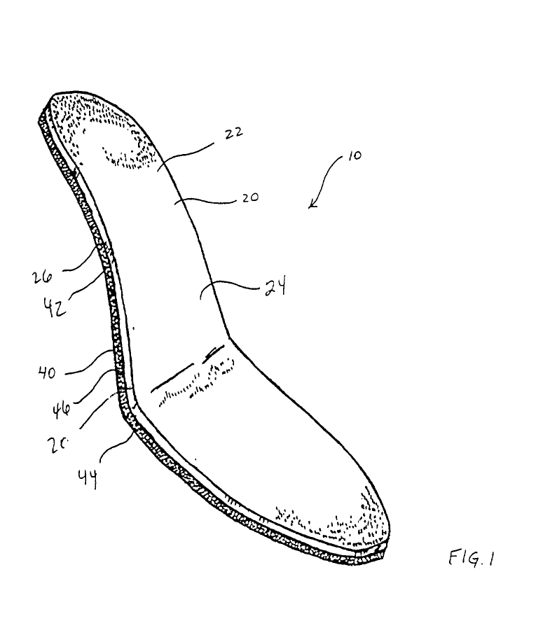

Referring now to FIG. 1, the present invention is a multi-layer deodorizer device 10 comprising first layer 20 and second layer 40. Preferably, first layer 20 is formed from filter paper 22 and is substantially flat, wherein the dimensional thickness preferably imparts at least minimal liquid absorbance characteristics. While filter paper 22 is the preferred material for first layer 20, one skilled in the art would readily recognize that other materials could be utilized such as, for exemplary purposes only, felt, blot paper, blot pad, cardboard, foam rubber, mesh, cellulose, neoprene, cloth or any other formed or fibrous material having appropriate absorbance characteristics.

Preferably, first layer 20 is impregnated with fragrance 30 (not shown). Fragrance 30 is preferably sprayed onto first layer 20, wherein fragrance 30 is preferably liquid. While a liquid fragrance is preferred, one skilled in the art would readily recognize that other types of fragrance could be utilized such as, for exemplary purposes only, gel, powder, beads or any other solid or semi-solid fragrance capable of being carried by multi-layer deodorizing device 10. In addition, one skilled in the art would readily recognize that other methods of fragrance application could be utilized such as, for exemplary purposes only, soaking or immersing first layer 20 in fragrance 30, squirting or brushing fragrance 30 onto first layer 20, or embedding gel, powder, beads or other solid or semi-solid fragrance into, onto or proximal to first layer 20.

Preferably, second layer 40 is substantially flat and substantially porous formed from charcoal. Preferably, first surface 42 of second layer 40 and second surface 26 of first layer 20 are secured together with adhesive, wherein first surface 24 of first layer 20 and second surface 44 of second layer are exposed. One skilled in the art would readily recognize that, while a two-layer arrangement is preferred, alternative layer configurations could be utilized such as, for exemplary purposes only, additional layers of fibrous material or porous material or combinations thereof. Moreover, while adhesive is the preferred method of securing first layer 20 and second layer 40, other methods could be utilized such as, for exemplary purposes only, stitching, heat-bonding, stapling or other appropriate method of annealing.

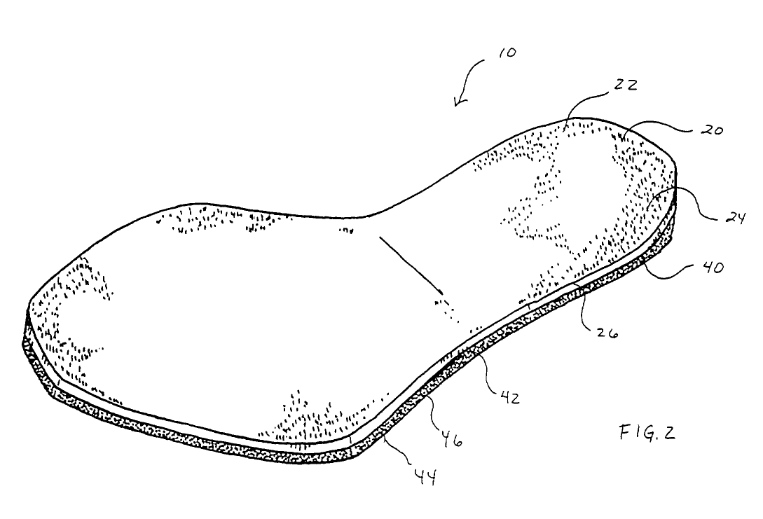

First layer 20 and second layer 40 of multi-layer deodorizing device 10 each define a shaped peripheral edge 26 and 46, wherein a pattern is preferably defined thereby such as, a preferable silhouette of a shoe. The defined shape of multi-layer deodorizing device 10 and the dimensions of first layer 20 and second layer 40 preferably enable placement of multi-layer deodorizing device 10 into a user-selected personal article or accessory. For example, it is contemplated that a generally shoe-shaped multi-layer deodorizing device 10 could be dimensioned, as best seen in FIG. 2, to fit within a man's shoe, or as best seen in FIG. 1, could be dimensioned to fit within a more narrow woman's shoe, wherein placement of multi-layer deodorizing device 10 within any such shoe would result in substantial contact between second layer 40 and the inner surface of the shoe. Such contact enables second layer 40 to function to remove odors from the inner surface of the shoe.

In an alternate embodiment, first layer 20 could be removably affixed to second layer 40, thereby enabling user-selectable use or reuse of a single layer.

In an alternate embodiment, first layer 20 could be fragrance-free.

In an alternate embodiment, first layer 20 could be substantially odor-absorbent and second layer 40 could be substantially fibrous.

In an alternate embodiment, multi-layer deodorizing device 10 could be treated using known procedures to provide glow-in-the-dark properties and coloration.

In an alternate embodiment, multi-layer deodorizing device 10 could carry indicia thereon such as, for exemplary purposes only, logo imprints, custom name imprints, licensed characters or any other appropriate printed indicia.

In an alternate embodiment, multi-layer deodorizing device 10 could include a plurality of layers.

In an alternate embodiment, multi-layer deodorizing device 10 could be formed or cut into a basic geometric shape instead of a specialized patterned shape.

In an alternate embodiment, multi-layer deodorizing device 10 could be formed into a general dome shape for specialized assistance in the deodorizing of a hat or other headgear.

In an alternate embodiment, multi-layer deodorizing device 10 could be formed into a generally elongated shape thereby enabling placement within a sock or other type of foot worn hosiery.

In an alternate embodiment, multi-layer deodorizing device 10 could be formed into a general hand shape thereby enabling placement within a glove or other type of hand wear.

In an alternate embodiment, multi-layer deodorizing device 10 could be non-disposable, wherein fragrance features and charcoal functionality could be refreshed.

In an alternate embodiment, porous second layer 40 could be formed from a non-charcoal filter such as, for exemplary purposes only, hepa, acetate or bacteriostatic filter.

In an alternate embodiment, porous second layer 40 could be formed from a variety of types of charcoal filters such as, for exemplary purposes only, activated carbon charcoal, granular activated carbon, silver charcoal or solid carbon block.

In an alternate embodiment, one multi-layer deodorizing device 10 could have a left-shoe shape, a second multi-layer deodorizing device 10 could have a right-shoe shape and the left and right shoe shaped devices could be linked together to facilitate placement and removal within a pair of shoes.

In an alternate embodiment, first layer 20 could be removably carried by second layer 40, wherein second layer 40 could be disposable and replaceable and first layer 20 could be washable and capable of fragrance refreshening or refilling.

In an alternate embodiment, multi-layer deodorizing device 10 could carry an elongated ribbon, strap, string or tab extending therefrom, thereby enabling quick removal from a user's shoe without reaching therein.

In use, multi-layer deodorizing device 10 aids in freshening and deodorizing a personal article, such as a shoe, wherein multi-layer deodorizing device 10 is placed within a user's shoe, preferably with second layer 40 generally in contact with the interior, foot-contacting surface of the shoe. Multi-layer deodorizing device 10 remains in the shoe during storage, resulting in dual freshening of the immediately surrounding storage area. To wear the refreshed shoe, a user removes multi-layer deodorizing device 10 therefrom.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. A multi-layer deodorizing device, comprising:
at least one substantially fibrous layer and at least one substantially porous layer, said at least one said substantially porous layer carried by said at least one substantially fibrous layer.

2. The multi-layer deodorizing device of claim 1, further comprising at least one extended member, said at least one extended member carried by said device and extending therefrom.

3. The multi-layer deodorizing device of claim 1, further comprising fragrance.

4. The multi-layer deodorizing device of claim 1, wherein said at least one substantially fibrous layer has at least minimal liquid absorbance characteristics.

5. The multi-layer deodorizing device of claim 1, wherein said at least one substantially fibrous layer is washable.

6. The multi-layer deodorizing device of claim 1, wherein said at least one substantially fibrous layer is disposable.

7. The multi-layer deodorizing device of claim 1, wherein said at least one substantially fibrous layer comprises rubber.

8. The multi-layer deodorizing device of claim 3, wherein said fragrance is liquid.

9. The multi-layer deodorizing device of claim 3, wherein said fragrance is at least semi-solid.

10. The multi-layer deodorizing device of claim 1, wherein said at least one substantially porous layer comprises charcoal.

11. The multi-layer deodorizing device of claim 1, wherein said at least one substantially porous layer is a filter.

12. The multi-layer deodorizing device of claim 1, wherein a first surface of said at least one substantially fibrous layer and a first surface of said at least one substantially porous layer are secured together with adhesive.

13. The multi-layer deodorizing device of claim 1, wherein a first surface of said at least one substantially fibrous layer and a first surface of said at least one substantially porous layer are removably carried by each other.

14. The multi-layer deodorizing device of claim 1, wherein said device is treated for coloration effects.

15. The multi-layer deodorizing device of claim 1, wherein said device carries indicia thereon.

16. A multi-layer, shaped deodorizing device comprising:
at least one absorbing layer having a shaped peripheral edge and
at least one filtering layer having a shaped peripheral edge, wherein said at least one absorbing layer and said at least one filtering layer are affixed in at least one position therebetween.

17. The multi-layer, shaped deodorizing device of claim 16, wherein said device is generally dome shaped.

18. The multi-layer, shaped deodorizing device of claim 16, wherein said device carries refreshable fragrance.

19. The multi-layer, shaped deodorizing device of claim 16, wherein said filtering layer has restorable properties.

20. A method of removing odors from a personal article or space comprising the steps of:
a) obtaining a multi-layer deodorizing device having at least one substantially fibrous, fragrance infused layer affixed to at least one substantially porous layer;
b) selecting a personal article for freshening;
c) placing said multi-layer deodorizing device into the personal article positioned wherein said substantially porous layer is in proximate to said personal article;
d) allowing said multi-layer deodorizing device to remain substantially within said personal article for a user-selected period of time;
e) removing said multi-layer deodorizing device from said personal article for use or wearing of said refreshed personal article.
